(19) United States
(12) Patent Application Publication
(10) Pub. No.: US 2011/0041589 A1
(43) Pub. Date: Feb. 24, 2011

(54) NEW HPCL METHOD

(75) Inventors: Erra Koteswara Satya Vijayakumar, Maharashtra (IN); Jagdish Chandramohan Arekar, Maharashtra (IN); Sachin Sahebrao Patil, Maharashtra (IN); Jagdish Dhonduram Dalvi, Maharashtra (IN); Dilip Manikchand Dhore, Maharashtra (IN)

Correspondence Address: FULBRIGHT & JAWORSKI L.L.P.;Attn: MN IP Docket, 600 Congress Avenue, Suite 2400, Austin, TX, 78701, US

(73) Assignees: GENERICS [UK] LIMITED, Potters Bar, Hertfordshire (GB); MYLAN INDIA PRIVATE LIMITED, Maharashtra (IN)

(21) Appl. No.: 12/747,947

(22) PCT Filed: Dec. 12, 2008
(86) PCT No.: PCT/GB2008/051186

§ 371 Date: Nov. 12, 2010

(30) Foreign Application Priority Data
Dec. 14, 2007 (IN) 1678/KOL/2007

(51) Int. Cl. G01N 30/00; G01N 27/447

(52) U.S. Cl. 73/61.52; 204/461

(57) ABSTRACT

The present invention relates to new HPLC methods for the analysis of the drug substance clopidogrel and related substances. In a first method the mobile phase comprises two or more liquids, and the relative concentration of the liquids is varied to a predetermined gradient. In a second method the mobile phase comprises a polar protic organic solvent, and the stationary phase comprises a gel. The present invention also relates to a method for analysing a substance, comprising the detection and optional quantification of one or more specific impurities.

### CROSS-REFERENCE TO RELATED APPLICATION(S)

This application is a Section 371 National Stage Application of International No. PCT/GB2008/051186, filed 12 Dec. 2008 and published as WO 2009/077784 A2 on 25 Jun. 2009, which claims priority from the IN Patent Application No. 1678/KOL/2007, filed 14 Dec. 2007, the contents of which are incorporated herein in their entirety for all purposes.

### FIELD OF THE INVENTION

The present invention relates to new HPLC methods for the analysis of the drug substance clopidogrel and related substances. In a first method the mobile phase comprises two or more liquids, and the relative concentration of the liquids is varied to a predetermined gradient. In a second method the mobile phase comprises a polar protic organic solvent, and the stationary phase comprises a gel. The present invention also relates to a method for analysing a substance, comprising the detection and optional quantification of one or more specific impurities.

### BACKGROUND OF THE INVENTION

In order to secure marketing approval for a pharmaceutical product, a manufacturer must submit detailed evidence to the appropriate regulatory authorities to prove that the product is suitable for release onto the market. It is, therefore, necessary to satisfy regulatory authorities that the product is acceptable for administration to humans and that the particular pharmaceutical composition, which is to be marketed, is free from impurities at the time of release and that it has acceptable storage stability.

Submissions to regulatory authorities must include analytical data which demonstrate that impurities are absent from the active pharmaceutical ingredient (API) at the time of manufacture, or are present only in acceptable levels, and that the storage stability of the pharmaceutical composition is acceptable.

The likely impurities in APIs and pharmaceutical compositions include residual quantities of synthetic precursors (intermediates), by-products which arise during synthesis of the API, residual solvents, isomers of the API (e.g. geometrical isomers, diastereomers or enantiomers), contaminants which are present in materials used in the synthesis of the API or in the preparation of the pharmaceutical composition, and unidentified adventitious substances. Other impurities which may appear on storage include degradants of the API, for instance formed by hydrolysis or oxidation.

The health authorities have very stringent standards and manufacturers must demonstrate that their product is relatively free from impurities or within acceptable limits and that these standards are reproducible for each batch of pharmaceutical product that is produced.

The tests that are required to demonstrate that the API or pharmaceutical compositions are safe and effective include a purity assay test, a related substances test, a content uniformity test and a dissolution test. The purity assay test determines the purity of the test product when compared to a standard of a known purity, while the related substances test is used to quantify all the impurities present in the product. The content uniformity test ensures that batches of product like a tablet contain a uniform amount of API and the dissolution test ensures that each batch of product has a consistent dissolution and release of the API.

The technique of choice for the analysis of an API or pharmaceutical composition (e.g. a tablet or capsule) is usually High Performance Liquid Chromatography (HPLC) coupled with a UV-Visible detector. The API and the impurities present, if any, are separated on the HPLC stationary phase and they can be detected and quantified using their response obtained from the UV-Visible detector.

HPLC is a chromatographic separation technique in which high-pressure pumps force the substance or mixture being analyzed together with a liquid solvent—mobile phase, also referred to as the eluent—through a separating column containing the stationary phase.

HPLC analysis may be performed in isocratic or gradient mode. In isocratic mode, the mobile phase composition is constant throughout. A gradient HPLC separation is carried out by a gradual change over a period of time in the percentage of the two or more solvents making up the mobile phase. The change in solvent is controlled by a mixer which mixes the solvents to produce the mobile phase prior to its passing through the column.

If a substance interacts strongly with the stationary phase, it remains in the column for a relatively long time, whereas a substance that does not interact as strongly with the stationary phase elutes out of the column sooner. Depending upon the strength of interactions, the various constituents of the analyte appear at the end of the separating column at different times, known as retention times, where they can be detected and quantified by means of a suitable detector, such as a UV-Visible detector.

Clopidogrel (I), chemically known as methyl (+)-(S)-α-2-(chlorophenyl)-6,7-dihydrothieno[3,2-c]pyridine-5(4H)-acetate, is a potent oral anti-platelet agent often used in the treatment of coronary artery disease, peripheral vascular disease and cerebrovascular disease. Clopidogrel is currently marketed as hydrogen sulfate salt of the D-isomer.

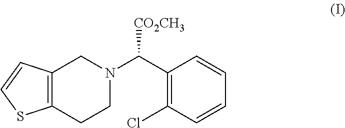

Several methods have been published in the literature to analyze clopidogrel, but these methods have not been primarily developed for the detection and quantitation of clopidogrel in bulk pharmaceutical preparations (see, for example, A. Mitakos et al. in J. Pharm. Biomed. Anal., 28 (3-4), 431-438, 2002; and Aboul-Enein et al. in J. Liquid Chromatography and Related Technologies, 28 (9), 1357-1365, 2005).

Additional HPLC methods have been reported in the literature, which have been developed for the analysis of clopidogrel or its metabolite in biological fluids (see, for example, E. Souri et al. in Biomedical Chromatography, 20 (12), 1309-1314, 2006; and A. Mitakos et al. in Anal. Chim. Acta, 505 (1), 107-114, 2004). HPLC methods suitable for the analysis of clopidogrel as API have been published by M. Semreen et al. in Int. J. Chem., 17 (2), 143-150, 2007. Additionally an official monograph on clopidogrel hydrogen sulfate appeared in US Pharmacopoeia 29, but a chiral HPLC method was employed to detect the impurities.

None of the current HPLC methods are suitable for the detection and quantification of all synthetic intermediates and other related substances that are present in a clopidogrel sample, particularly a sample synthesized by the route disclosed in European Patent No. EP 1 353 928. Current methods are also deficient in estimating the total impurities in clopidogrel and its salts.

Therefore, the HPLC methods reported in the prior art are not particularly convenient or suitable for analyzing clopidogrel and its salts as an API, particularly with respect to related substances.

Consequently, although several HPLC methods have been reported in the literature for the analysis of clopidogrel and/or its salts and its impurities, there is still a need for an alternative method which avoids the problems associated with the known methods as discussed above.

### SUMMARY OF THE INVENTION

The present invention provides validation of a new, efficient, reproducible and simple HPLC method for the analysis of clopidogrel, particularly with respect to the related substances formed during the synthesis.

The present invention also provides a new, alternative method for analyzing clopidogrel, its impurities and related substances, whilst avoiding the typical problems associated with the prior art methods.

In one particular aspect, the present invention provides a new, accurate and sensitive HPLC method for the detection and quantitation of intermediates and related substances that are formed and may remain in batches of clopidogrel and/or its salts synthesized by the route disclosed in European Patent No. EP 1 353 928.

The term “clopidogrel” as used herein throughout the description and claims means clopidogrel and/or any salt, solvate, isomer or enantiomer thereof. The current invention is particularly useful for the analysis of clopidogrel free base, clopidogrel bisulfate, clopidogrel hydrogen bromide, clopidogrel mesylate, clopidogrel besylate, clopidogrel tosylate, clopidogrel naphthalene-2-sulfonate(napsylate), clopidogrel naphthalene-1,5-disulfonate, clopidogrel oxalate, clopidogrel L-tartrate or clopidogrel D-tartrate.

A first aspect of the current invention provides a HPLC method for analyzing clopidogrel, wherein the mobile phase comprises two or more liquids, including a first liquid A and a second liquid B, and the relative concentration of the liquids is varied to a predetermined gradient.

Preferably the first liquid A is aqueous based, such as water or an aqueous solution of a buffer.

Preferably, the buffer is an acid or an organic salt or an inorganic salt.

Typically, the buffer is a phosphate salt, an acetate salt, a formate salt or trifluoroacetic acid. Most preferably, the buffer is a phosphate salt, such as potassium dihydrogen phosphate (optionally anhydrous).

The buffer can be present at a concentration of 0.001 to 0.1 M, preferably at a concentration of 0.001 to 0.05 M, more preferably at a concentration of 0.005 to 0.05 M, most preferably at a concentration of approximately 0.02 M.

Preferably the buffer is potassium dihydrogen phosphate (optionally anhydrous) present at a concentration of 0.005 to 0.05 M. Most preferably, the buffer is potassium dihydrogen phosphate (optionally anhydrous) present at a concentration of approximately 0.02 M.

Preferably, the pH of the buffer is approximately 2 to 6, more preferably the pH is between 2.5 and 4.5, most preferably the pH of the buffer is approximately 3.5.

Typically, the method of the first aspect of the current invention is carried out at a column temperature between approximately 15 to 40° C.

The first liquid A may contain one or more additional solvent(s) which are preferably substantially water-miscible.

As used herein in relation to any aspect of the present invention, the term “substantially miscible” in relation to two liquids X and Y means that when mixed together at 20° C. and 1 atmosphere pressure, X and Y form a single phase between two mole fractions of Y, x_{Y1} and x_{Y2}, wherein the magnitude of Δx_{Y} (=x_{Y2}−x_{Y1}) is at least 0.05. For example, X and Y may form a single phase where the mole fraction of Y, x_{Y}, is from 0.40 to 0.45, or from 0.70 to 0.75; in both cases Δx_{Y}=0.05. Preferably, the magnitude of Δx_{Y} is at least 0.10, more preferably at least 0.25, more preferably at least 0.50, more preferably at least 0.75, more preferably at least 0.90, even more preferably at least 0.95. Most preferably the term “substantially miscible” in relation to two liquids X and Y means that when mixed together at 20° C. and 1 atmosphere pressure, X and Y form a single phase when mixed together in any proportion.

In one embodiment the additional solvent is an organic solvent selected from a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof. Preferably the additional solvent is selected from methanol, ethanol, acetonitrile, n-propanol or iso-propanol, or a mixture thereof. The additional solvent in the first liquid A may or may not be the same solvent as the second liquid B. The additional solvent in the first liquid A is preferably methanol.

In another embodiment the first liquid A comprises 10 to 90% v/v, preferably 30 to 80% v/v, more preferably 50 to 70% v/v of the additional solvent. Most preferably the first liquid A comprises approximately 60% v/v of the additional solvent.

The second liquid B is preferably an organic solvent, such as methanol, ethanol, acetonitrile, n-propanol or iso-propanol, or a mixture thereof. Most preferably, the second liquid is methanol.

In one embodiment of the first aspect of the current invention the second liquid B is a substantially water-miscible solvent.

Preferably the second liquid B is a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof.

A preferred embodiment of the first aspect of the current invention is when the first liquid A is a mixture of aqueous potassium dihydrogen phosphate (optionally anhydrous)-methanol (40:60 v/v) and the second liquid B is methanol.

Preferably a mobile phase flow rate of between 0.01 and 10 ml/min is used, more preferably a mobile phase flow rate of between 0.1 and 4 ml/min is used, more preferably a mobile phase flow rate of about 1 ml/min is used.

The method of the first aspect of the current invention may comprise a gradient programming so that the relative concentration of the liquids A and B is varied to a gradient between 100% A:0% B to 0% A:100% B over a period of 10 to 180 minutes. Preferably, the gradient is between 100% A:0% B to 0% A:100% B over a period of 25 or 30 to 120 minutes, more preferably, 100% A:0% B to 0% A:100% B over a period of 25 or 30 to 60 minutes.

As used herein in relation to any aspect of the present invention, unless stated otherwise all percentages given in relation to the concentration of liquids A and/or B refer to the percentage by volume.

Alternatively, the first aspect of the current invention may comprise a gradient programming so that the relative concentration of the liquids A and B is varied to a gradient from about 100% A:0% B, or from about 95% A:5% B, or from about 90% A:10% B, or from about 85% A:15% B, to about 100% A:0% B, or to about 5% A:95% B, or to about 10% A:90% B, or to about 15% A:85% B, or to about 50% A:50% B. The variation in gradient may typically take place over 10 to 180 minutes, preferably over 30 to 120 minutes, more preferably over 30 to 60 minutes.

A particularly preferred embodiment of the first aspect of the current invention is when the first liquid A is 0.02 M aqueous potassium dihydrogen phosphate (optionally anhydrous)-methanol (40:60 v/v) and the second liquid B is methanol.

A particularly preferred method according to the first aspect of the current invention is when the first liquid A is 0.02 M aqueous potassium dihydrogen phosphate (optionally anhydrous)-methanol (40:60 v/v) and the second liquid B is methanol and the gradient is as follows:

| Time (min) | % A | % B |
|---|---|---|
| 0 | 100 | 0 |
| 5 | 100 | 0 |
| 35 | 50 | 50 |
| 40 | 100 | 0 |
| 50 | 100 | 0 |

In one embodiment of the first aspect of the current invention the stationary phase used is a gel, preferably a silica gel.

In another embodiment, the stationary phase used is chiral and/or the mobile phase further comprises a chiral selector.

Preferably, the stationary phase used in the first aspect of the current invention is reverse phase such as octadecylsilyl silica gel, octylsilyl silica gel, phenylalkyl silica gel, cyanopropyl silica gel, aminopropyl silica gel or an alkyl-diol silica gel. Particularly suitable stationary phases include octadecylsilyl silica gel or octylsilyl silica gel. A particularly preferred stationary phase comprises a Sunfire C18 (250 mm×4.6 mm), 5 μm column, preferably with a 100 Å pore size.

Preferably the stationary phase has a particle size of between 0.1 and 100 μm, or between 0.5 and 25 μm, or between 1 and 10 μm. More preferably the stationary phase has a particle size of about 5 μm.

Preferably the stationary phase has a pore size of between 10 and 1000 Å, or between 20 and 400 Å, or between 50 and 150 Å. More preferably the stationary phase has a pore size of about 100 Å.

In one embodiment of the first aspect of the current invention, the chromatography is carried out in a column between 10 mm and 5000 mm in length, or in a column between 50 mm and 1000 mm in length, or between 100 mm and 500 mm in length. More preferably the chromatography is carried out in a column about 250 mm in length.

The chromatography may be carried out in a column between 0.01 mm and 100 mm in internal diameter, or between 0.1 mm and 50 mm in internal diameter, or between 1 mm and 10 mm in internal diameter. More preferably the chromatography is carried out in a column about 4.6 mm in internal diameter.

The eluent may be analysed by a detector such as a UV or visible spectrophotometer, a fluorescence spectrophotometer, a differential refractometer, an electrochemical detector, a mass spectrometer, a light scattering detector or a radioactivity detector.

In one embodiment of the first aspect of the current invention, the clopidogrel analysed is for use in a pharmaceutical composition. Preferably the method is a method of analysing a pharmaceutical composition comprising clopidogrel.

In another embodiment of the first aspect of the current invention, the clopidogrel is in the form of a salt, solvate or hydrate. Preferably the clopidogrel is either the bisulfate or hydrogen bromide salt.

In one embodiment of the first aspect of the current invention, the HPLC method detects and optionally quantifies one or more impurities selected from:
(+)-(S)-(o-chlorophenyl)-6,7-dihydrothieno[3,2-c]pyridine-5(4H)-acetic acid (II);
methyl (±)-(o-chlorophenyl)-4,5-dihydrothieno[2,3-c]pyridine-6(7H)-acetate (III);
D-(+)-α-4,5,6,7-tetrahydrothieno[3,2-c]-5-pyridyl-(o-chlorophenyl)acetamide (IV); and
α-4,5,6,7-tetrahydrothieno[3,2-c]-5-pyridyl-(o-chlorophenyl)acetonitrile (V).

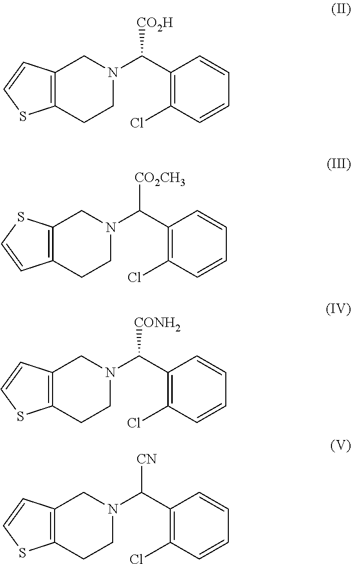

Preferably the HPLC method according to the first aspect of the current invention detects and optionally quantifies in a single run one or more impurities selected from:
(+)-(S)-(o-chlorophenyl)-6,7-dihydrothieno[3,2-c]pyridine-5(4H)-acetic acid (II);
methyl (±)-(o-chlorophenyl)-4,5-dihydrothieno[2,3-c]pyridine-6(7H)-acetate (III);
D-(+)-α-4,5,6,7-tetrahydrothieno[3,2-c]-5-pyridyl-(o-chlorophenyl)acetamide (IV); and
α-4,5,6,7-tetrahydrothieno[3,2-c]-5-pyridyl-(o-chlorophenyl)acetonitrile (V).

Most preferably the HPLC method according to the first aspect of the current invention efficiently detects and quantifies in a single run all impurities including those selected from the following compounds:
(+)-(S)-(o-chlorophenyl)-6,7-dihydrothieno[3,2-c]pyridine-5(4H)-acetic acid (II) (listed as Impurity A in USP 29);
methyl (±)-(o-chlorophenyl)-4,5-dihydrothieno[2,3-c]pyridine-6(7H)-acetate (III) (listed as Impurity B in USP 29);
D-(+)-α-4,5,6,7-tetrahydrothieno[3,2-c]-5-pyridyl-(o-chlorophenyl)acetamide (IV); and
α-4,5,6,7-tetrahydrothieno[3,2-c]-5-pyridyl-(o-chlorophenyl)acetonitrile (V).

In any of the above embodiments of the first aspect of the current invention, the detection and/or quantification of impurity (II) and/or (IV) may instead or in addition comprise the detection and/or quantification of the enantiomer of impurity (II) and/or (IV). Furthermore, the detection and/or quantification of impurity (II) and/or (IV) may optionally instead comprise the detection and/or quantification of both enantiomers of impurity (II) and/or (IV) without distinguishing between them.

Also, in any of the above embodiments of the first aspect of the current invention, the detection and/or quantification of impurity (III) and/or (V) may instead or in addition comprise the detection and/or quantification of one or more specific enantiomers of impurity (III) and/or (V).

A second aspect of the current invention provides a HPLC method for analysing clopidogrel, wherein the mobile phase comprises a polar protic organic solvent, and the stationary phase comprises a gel.

Preferably the polar protic organic solvent is a substantially water-miscible solvent.

In one embodiment of the second aspect of the current invention, the polar protic organic solvent is selected from acetic acid, methanol, ethanol, n-propanol, n-butanol, iso-propanol, iso-butanol, sec-butanol or tert-butanol, or a mixture thereof. Preferably the polar protic organic solvent is selected from methanol, ethanol, n-propanol or iso-propanol, or a mixture thereof. Most preferably the polar protic organic solvent is methanol.

In another embodiment of the second aspect of the current invention, the mobile phase comprises two or more liquids, including a first liquid A and a second liquid B, and the second liquid B comprises or is the polar protic organic solvent.

Preferably the first liquid A is aqueous based, such as water or an aqueous solution of a buffer.

Preferably the buffer is an acid or an organic salt or an inorganic salt.

Typically the buffer is a phosphate salt, an acetate salt, a formate salt or trifluoroacetic acid. Most preferably the buffer is a phosphate salt, such as potassium dihydrogen phosphate.

The buffer can be present at a concentration of 0.001 to 0.1 M, preferably at a concentration of 0.001 to 0.05 M, more preferably at a concentration of 0.005 to 0.05 M, most preferably at a concentration of approximately 0.02 M.

Preferably the buffer is potassium dihydrogen phosphate present at a concentration of 0.005 to 0.05 M. Most preferably, the buffer is potassium dihydrogen phosphate present at a concentration of approximately 0.02 M.

Preferably, the pH of the buffer is approximately 2 to 6, more preferably the pH is between 2.5 and 4.5, most preferably the pH of the buffer is approximately 3.5.

The first liquid A may optionally comprise one or more additional solvents, which are preferably substantially water-miscible.

The additional solvent may be an organic solvent selected from a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof. Preferably the additional solvent is selected from methanol, ethanol, acetonitrile, n-propanol or iso-propanol, or a mixture thereof. The additional solvent in the first liquid A may or may not be the same solvent as the second liquid B. The additional solvent in the first liquid A is preferably methanol.

The first liquid A may comprise 10 to 90% v/v, preferably 30 to 80% v/v, more preferably 50 to 70% v/v of the additional solvent. Most preferably the first liquid A comprises approximately 60% v/v of the additional solvent.

In a particularly preferred embodiment the first liquid A is a mixture of aqueous potassium dihydrogen phosphate-methanol (40:60 v/v) and the second liquid B is methanol.

Preferably a mobile phase flow rate of between 0.01 and 10 ml/min is used, more preferably a mobile phase flow rate of between 0.1 and 4 ml/min is used, more preferably a mobile phase flow rate of about 1 ml/min is used.

In one embodiment of the second aspect of the current invention, the HLPC method is an isocratic method, preferably such that the relative concentration of the liquids A and B is set between 99.5% A:0.5% B and 0.5% A:99.5% B, or between 90% A:10% B and 10% A:90% B, more preferably between 75% A:25% B and 25% A:75% B. More preferably the relative concentration of the liquids A and B is about 50% A:50% B.

In an alternative embodiment of the second aspect of the current invention, the relative concentration of the liquids of the mobile phase is varied to a predetermined gradient. Typically, the method may comprise a gradient programming so that the relative concentration of the liquids A and B is varied to a gradient between 100% A:0% B to 0% A:100% B over a period of 10 to 180 minutes. Preferably, the gradient is between 100% A:0% B to 0% A:100% B over a period of 30 to 120 minutes, more preferably, 100% A:0% B to 0% A:100% B over a period of 30 to 60 minutes. Alternatively, a gradient programming may be used so that the relative concentration of the liquids A and B is varied to a gradient from about 100% A:0% B, or from about 95% A:5% B, or from about 90% A:10% B, or from about 85% A:15% B, to about 100% A:0% B, or to about 5% A:95% B, or to about 10% A:90% B, or to about 15% A:85% B, or to about 50% A:50% B. The variation in gradient may typically take place over 10 to 180 minutes, preferably over 30 to 120 minutes, more preferably over 30 to 60 minutes.

In a preferred embodiment of the second aspect of the current invention, the first liquid A is 0.02 M aqueous potassium dihydrogen phosphate-methanol (40:60 v/v) and the second liquid B is methanol. Preferably in such an embodiment the gradient is as follows:

| Time (min) | % A | % B |
|---|---|---|
| 0 | 100 | 0 |
| 5 | 100 | 0 |
| 35 | 50 | 50 |
| 40 | 100 | 0 |
| 50 | 100 | 0 |

In one embodiment of the second aspect of the current invention the stationary phase used is a silica gel.

In another embodiment, the stationary phase used is chiral and/or the mobile phase further comprises a chiral selector.

Preferably, the stationary phase used in the second aspect of the current invention is reverse phase such as octadecylsilyl silica gel, octylsilyl silica gel, phenylalkyl silica gel, cyanopropyl silica gel, aminopropyl silica gel or an alkyl-diol silica gel. Particularly suitable stationary phases include octadecylsilyl silica gel or octylsilyl silica gel. A particularly preferred stationary phase comprises a Sunfire C18 (250 mm×4.6 mm), 5 μm column, preferably with a 100 Å pore size.

Preferably the stationary phase has a particle size of between 0.1 and 100 μm, or between 0.5 and 25 μm, or between 1 and 10 μm. More preferably the stationary phase has a particle size of about 5 μm.

Preferably the stationary phase has a pore size of between 10 and 1000 Å, or between 20 and 400 Å, or between 50 and 150 Å. More preferably the stationary phase has a pore size of about 100 Å.

Preferably the chromatography is carried out at a temperature between approximately 15 to 40° C.

In one embodiment of the second aspect of the current invention, the chromatography is carried out in a column between 10 mm and 5000 mm in length, or in a column between 50 mm and 1000 mm in length, or between 100 mm and 500 mm in length. More preferably the chromatography is carried out in a column about 250 mm in length.

The chromatography may be carried out in a column between 0.01 mm and 100 mm in internal diameter, or between 0.1 mm and 50 mm in internal diameter, or between 1 mm and 10 mm in internal diameter. More preferably the chromatography is carried out in a column about 4.6 mm in internal diameter.

The eluent may be analysed by a detector such as a UV or visible spectrophotometer, a fluorescence spectrophotometer, a differential refractometer, an electrochemical detector, a mass spectrometer, a light scattering detector or a radioactivity detector.

In one embodiment of the second aspect of the current invention, the clopidogrel analysed is for use in a pharmaceutical composition. Preferably the method is a method of analysing a pharmaceutical composition comprising clopidogrel.

In another embodiment of the second aspect of the current invention, the clopidogrel is in the form of a salt, solvate or hydrate. Preferably the clopidogrel is either the bisulfate or hydrogen bromide salt.

In one embodiment of the second aspect of the current invention, the HPLC method detects and optionally quantifies one or more impurities selected from:
(+)-(S)-(o-chlorophenyl)-6,7-dihydrothieno[3,2-c]pyridine-5(4H)-acetic acid (II);
methyl (±)-(o-chlorophenyl)-4,5-dihydrothieno[2,3-c]pyridine-6(7H)-acetate (III);
D-(+)-α-4,5,6,7-tetrahydrothieno[3,2-c]-5-pyridyl-(o-chlorophenyl)acetamide (IV); and
α-4,5,6,7-tetrahydrothieno[3,2-c]-5-pyridyl-(o-chlorophenyl)acetonitrile (V).

Preferably the HPLC method according to the second aspect of the current invention detects and optionally quantifies in a single run one or more impurities selected from:
(+)-(S)-(o-chlorophenyl)-6,7-dihydrothieno[3,2-c]pyridine-5(4H)-acetic acid (II);
methyl (±)-(o-chlorophenyl)-4,5-dihydrothieno[2,3-c]pyridine-6(7H)-acetate (III);
D-(+)-α-4,5,6,7-tetrahydrothieno[3,2-c]-5-pyridyl-(o-chlorophenyl)acetamide (IV); and
α-4,5,6,7-tetrahydrothieno[3,2-c]-5-pyridyl-(o-chlorophenyl)acetonitrile (V).

Most preferably the HPLC method according to the second aspect of the current invention detects and quantifies in a single run all impurities including those selected from the following compounds:
(+)-(S)-(o-chlorophenyl)-6,7-dihydrothieno[3,2-c]pyridine-5(4H)-acetic acid (II);
methyl (±)-(o-chlorophenyl)-4,5-dihydrothieno[2,3-c]pyridine-6(7H)-acetate (III);
D-(+)-α-4,5,6,7-tetrahydrothieno[3,2-c]-5-pyridyl-(o-chlorophenyl)acetamide (IV); and
α-4,5,6,7-tetrahydrothieno[3,2-c]-5-pyridyl-(o-chlorophenyl)acetonitrile (V).

In any of the above embodiments of the second aspect of the current invention, the detection and/or quantification of impurity (II) and/or (IV) may instead or in addition comprise the detection and/or quantification of the enantiomer of impurity (II) and/or (IV). Furthermore, the detection and/or quantification of impurity (II) and/or (IV) may optionally instead comprise the detection and/or quantification of both enantiomers of impurity (II) and/or (IV) without distinguishing between them.

Also, in any of the above embodiments of the second aspect of the current invention, the detection and/or quantification of impurity (III) and/or (V) may instead or in addition comprise the detection and/or quantification of one or more specific enantiomers of impurity (III) and/or (V).

A third aspect of the current invention provides a method for analysing a substance, comprising the detection and optional quantification of one or more impurities selected from:
D-(+)-α-4,5,6,7-tetrahydrothieno[3,2-c]-5-pyridyl-(o-chlorophenyl)acetamide (IV); and
α-4,5,6,7-tetrahydrothieno[3,2-c]-5-pyridyl-(o-chlorophenyl)acetonitrile (V).

Preferably, the method of the third aspect of the current invention further comprises the detection and optional quantification of one or more impurities selected from:
(+)-(S)-(o-chlorophenyl)-6,7-dihydrothieno[3,2-c]pyridine-5(4H)-acetic acid (II); and
methyl (±)-(o-chlorophenyl)-4,5-dihydrothieno[2,3-c]pyridine-6(7H)-acetate (III).

In any of the above embodiments of the third aspect of the current invention, the detection and/or quantification of impurity (II) and/or (IV) may instead or in addition comprise the detection and/or quantification of the enantiomer of impurity (II) and/or (IV). Furthermore, the detection and/or quantification of impurity (II) and/or (IV) may optionally instead comprise the detection and/or quantification of both enantiomers of impurity (II) and/or (IV) without distinguishing between them.

Also, in any of the above embodiments of the third aspect of the current invention, the detection and/or quantification of impurity (III) and/or (V) may instead or in addition comprise the detection and/or quantification of one or more specific enantiomers of impurity (III) and/or (V).

In one embodiment of the third aspect of the present invention, the substance is an active pharmaceutical ingredient. Preferably the substance is clopidogrel, optionally in the form of a salt, solvate or hydrate. Most preferably the clopidogrel is either the bisulfate or hydrogen bromide salt. Preferably the clopidogrel analysed is for use in a pharmaceutical composition.

In one embodiment of the third aspect of the current invention, the method is a method of analysing a pharmaceutical composition comprising clopidogrel.

In another embodiment of the third aspect of the current invention, the substance comprises less than 25 wt. % of the one or more impurities. Preferably, the substance comprises less than 10 wt. %, less than 5 wt. % or less than 2 wt. % of the one or more impurities. More preferably the substance comprises less than 1 wt. %, or less than 0.5 wt. % of the one or more impurities.

In another embodiment of the third aspect of the current invention, the method comprises the use of HLPC, preferably such that the mobile phase comprises two or more liquids, including a first liquid A and a second liquid B.

Preferably, the first liquid A is aqueous based, such as water or an aqueous solution of a buffer.

Preferably, the buffer is an acid or an organic salt or an inorganic salt.

Typically the buffer is a phosphate salt, an acetate salt, a formate salt or trifluoroacetic acid. Most preferably the buffer is a phosphate salt, such as potassium dihydrogen phosphate.

The buffer can be present at a concentration of 0.001 to 0.1 M, preferably at a concentration of 0.001 to 0.05 M, more preferably at a concentration of 0.005 to 0.05 M, most preferably at a concentration of approximately 0.02 M.

Preferably the buffer is potassium dihydrogen phosphate present at a concentration of 0.005 to 0.05 M. Most preferably, the buffer is potassium dihydrogen phosphate present at a concentration of approximately 0.02 M.

Preferably, the pH of the buffer is approximately 2 to 6, more preferably the pH is between 2.5 and 4.5, most preferably the pH of the buffer is approximately 3.5.

The first liquid A may optionally comprise one or more additional solvents, which are preferably substantially water-miscible.

The additional solvent may be an organic solvent selected from a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof. Preferably the additional solvent is selected from methanol, ethanol, acetonitrile, n-propanol or iso-propanol, or a mixture thereof. The additional solvent in the first liquid A may or may not be the same solvent as the second liquid B. The additional solvent in the first liquid A is preferably methanol.

The first liquid A may comprise 10 to 90% v/v, preferably 30 to 80% v/v, more preferably 50 to 70% v/v of the additional solvent. Most preferably the first liquid A comprises approximately 60% v/v of the additional solvent.

The second liquid B is preferably an organic solvent, such as methanol, ethanol, acetonitrile, n-propanol or iso-propanol, or a mixture thereof.

Preferably the second liquid B is a substantially water-miscible solvent.

Preferably the second liquid B is a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof. Most preferably the second liquid B is methanol.

In a particularly preferred embodiment the first liquid A is a mixture of aqueous potassium dihydrogen phosphate-methanol (40:60 v/v) and the second liquid B is methanol.

Preferably a mobile phase flow rate of between 0.01 and 10 ml/min is used, more preferably a mobile phase flow rate of between 0.1 and 4 ml/min is used, more preferably a mobile phase flow rate of about 1 ml/min is used.

In one embodiment of the third aspect of the current invention, the HLPC method is an isocratic method, preferably such that the relative concentration of the liquids A and B is set between 99.5% A:0.5% B and 0.5% A:99.5% B, or between 90% A:10% B and 10% A:90% B, more preferably between 75% A:25% B and 25% A:75% B. More preferably the relative concentration of the liquids A and B is about 50% A:50% B.

In an alternative embodiment of the third aspect of the current invention, the relative concentration of the liquids of the mobile phase is varied to a predetermined gradient. Typically, the method may comprise a gradient programming so that the relative concentration of the liquids A and B is varied to a gradient between 100% A:0% B to 0% A:100% B over a period of 10 to 180 minutes. Preferably, the gradient is between 100% A:0% B to 0% A:100% B over a period of 30 to 120 minutes, more preferably, 100% A:0% B to 0% A:100% B over a period of 30 to 60 minutes. Alternatively, a gradient programming may be used so that the relative concentration of the liquids A and B is varied to a gradient from about 100% A:0% B, or from about 95% A:5% B, or from about 90% A:10% B, or from about 85% A:15% B, to about 100% A:0% B, or to about 5% A:95% B, or to about 10% A:90% B, or to about 15% A:85% B, or to about 50% A:50% B. The variation in gradient may typically take place over 10 to 180 minutes, preferably over 30 to 120 minutes, more preferably over 30 to 60 minutes.

In a preferred embodiment of the third aspect of the current invention, the first liquid A is 0.02 M aqueous potassium dihydrogen phosphate-methanol (40:60 v/v) and the second liquid B is methanol. Preferably in such an embodiment the gradient is as follows:

| Time (min) | % A | % B |
|---|---|---|
| 0 | 100 | 0 |
| 5 | 100 | 0 |
| 35 | 50 | 50 |
| 40 | 100 | 0 |
| 50 | 100 | 0 |

In one embodiment of the third aspect of the current invention the stationary phase used is a gel, preferably a silica gel.

In another embodiment, the stationary phase used is chiral and/or the mobile phase further comprises a chiral selector.

Preferably, the stationary phase used in the third aspect of the current invention is reverse phase such as octadecylsilyl silica gel, octylsilyl silica gel, phenylalkyl silica gel, cyanopropyl silica gel, aminopropyl silica gel or an alkyl-diol silica gel. Particularly suitable stationary phases include octadecylsilyl silica gel or octylsilyl silica gel. A particularly preferred stationary phase comprises a Sunfire C18 (250 mm×4.6 mm), 5 μm column, preferably with a 100 Å pore size.

Preferably the stationary phase has a particle size of between 0.1 and 100 μm, or between 0.5 and 25 μm, or between 1 and 10 μm. More preferably the stationary phase has a particle size of about 5 μm.

Preferably the stationary phase has a pore size of between 10 and 1000 Å, or between 20 and 400 Å, or between 50 and 150 Å. More preferably the stationary phase has a pore size of about 100 Å.

Preferably the chromatography is carried out at a temperature between approximately 15 to 40° C.

In one embodiment of the third aspect of the current invention, the chromatography is carried out in a column between 10 mm and 5000 mm in length, or in a column between 50 mm and 1000 mm in length, or between 100 mm and 500 mm in length. More preferably the chromatography is carried out in a column about 250 mm in length.

The chromatography may be carried out in a column between 0.01 mm and 100 mm in internal diameter, or between 0.1 mm and 50 mm in internal diameter, or between 1 mm and 10 mm in internal diameter. More preferably the chromatography is carried out in a column about 4.6 mm in internal diameter.

The eluent may be analysed by a detector such as a UV or visible spectrophotometer, a fluorescence spectrophotometer, a differential refractometer, an electrochemical detector, a mass spectrometer, a light scattering detector or a radioactivity detector.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

### DETAILED DESCRIPTION

The current invention can be used to analyse clopidogrel and/or its salts as an API or clopidogrel and/or its salts when prepared as a pharmaceutical composition.

The pharmaceutical compositions that can be analysed by the current invention include solid and liquid compositions and optionally comprise one or more pharmaceutically acceptable carriers or excipients. Solid form compositions include powders, tablets, pills, capsules, cachets, suppositories, and dispersible granules. Liquid compositions include solutions or suspensions which can be administered by oral, injectable or infusion routes.

The term “impurities” or “related substances” as used herein throughout the specification can mean either impurities formed in the manufacture of the API or the pharmaceutical composition and/or formed by degradation of the API or in the pharmaceutical composition on storage.

As discussed above, the HPLC methods reported in the prior art are not suitable for analysing clopidogrel, particularly with respect to the related substances formed in the synthesis of clopidogrel and/or its salts prepared by the process disclosed in European Patent No. EP 1 353 928. A reason for the difficulties encountered in the prior art could be due to the large polarity differences between the related substances and clopidogrel.

However, a particularly preferred embodiment of the current invention solves this problem and efficiently detects and quantifies, in a single run, all impurities and intermediates formed in this particular synthetic process. The present invention is advantageous as the gradient method allows the elution of all polar to non-polar impurities.

The current invention is also advantageous as the method is selective, linear, precise, accurate and robust for the analysis of related substances in clopidogrel and/or its salts. In addition, the current invention is highly sensitive and allows detection and quantification of related substances in clopidogrel and/or its salts at levels much lower than acceptance limits specified by health authorities.

In addition, the method of the current invention can be used to easily detect and quantify all degradation impurities formed on storage of samples of clopidogrel. This was established by carrying out forced degradation studies as per ICH Q1A Guidelines and validated as per ICH Q2A Guidelines covering the parameters Specificity, Linearity and Range, Precision (Repeatability, Reproducibility and Intermediate Precision), Accuracy, Limit of Detection (LOD), Limit of Quantitation (LOQ), Robustness and System Suitability.

The buffer optionally used in the first liquid A can be an inorganic salt such as sodium, potassium, calcium, magnesium, lithium or aluminium salts of phosphate, acetate or formate and mixtures thereof. Alternatively the buffer can be an organic salt such as the ammonium salt of acetate or formate and mixtures thereof. Alternatively the buffer can be a mineral acid or a carboxylic acid, such as acetic acid or trifluoroacetic acid. Preferably the first liquid A is a mixture of 0.02 M aqueous potassium dihydrogen phosphate (optionally anhydrous)-methanol (40:60 v/v).

The organic solvent(s) used as the additional solvent in liquid A or as the second liquid B can be organic solvents like lower alkyl alcohols, such as methanol, ethanol, n-propanol, butanol or iso-propanol, or mixtures thereof. Alternatively, the organic solvent(s) may be tetrahydrofuran or acetonitrile or any suitable organic solvent(s). Preferably the organic solvent is methanol.

Preferably the stationary phase used in the method of the current invention is selected from octadecylsilyl silica gel (RP-18) or octylsilyl silica gel (RP-8).

An internal standard reference compound may be used in the method of the current invention if required. Alternatively the concentration of the components analysed may be determined by comparison with one or more external reference compounds.

The inventors have tested the methods of the current invention extensively to show that they are reproducible, accurate, precise, linear with respect to concentration and robust.

While the present invention has been described in terms of its specific embodiments, certain modifications and equivalents will be apparent to those skilled in the art and are intended to be included within the scope of the present invention.

The methods of the invention disclosed herein can also be used for the analysis of compounds with similar chemical structures and/or similar chemical or physical properties to clopidogrel, such as ticlopidine, and their salts and/or isomers or enantiomers.

The following paragraphs enumerated consecutively from 1 through 179 provide for various aspects of the present invention. In one embodiment, the present invention provides:

1. A HPLC method for analysing clopidogrel, wherein the mobile phase comprises two or more liquids, including a first liquid A and a second liquid B, and the relative concentration of the liquids is varied to a predetermined gradient.
2. A HPLC method according to paragraph 1, wherein the first liquid A is aqueous based.
3. A HPLC method according to paragraph 2, wherein the first liquid A comprises water or an aqueous solution of a buffer.
4. A HPLC method according to paragraph 3, wherein the buffer is an acid or an organic salt or an inorganic salt.
5. A HPLC method according to paragraph 4, wherein the buffer is a phosphate salt, an acetate salt, a formate salt or trifluoroacetic acid.
6. A HPLC method according to paragraph 4 or 5, wherein the buffer is a phosphate salt.
7. A HPLC method according to paragraph 6, wherein the buffer is potassium dihydrogen phosphate.
8. A HPLC method according to any one of paragraphs 3 to 7, wherein the buffer is present at a concentration of 0.001 to 0.1 M.
9. A HPLC method according to paragraph 8, wherein the buffer is present at a concentration of 0.001 to 0.05 M.
10. A HPLC method according to paragraph 9, wherein the buffer is present at a concentration of 0.005 to 0.05 M.
11. A HPLC method according to paragraph 10, wherein the buffer is present at a concentration of approximately 0.02 M.
12. A HPLC method according to paragraph 10, wherein the buffer is potassium dihydrogen phosphate present at a concentration of 0.005 to 0.05 M.
13. A HPLC method according to paragraph 12, wherein the potassium dihydrogen phosphate is present at a concentration of approximately 0.02 M.
14. A HPLC method according to any one of paragraphs 3 to 13, wherein the pH of the buffer is approximately 2 to 6.
15. A HPLC method according to paragraph 14, wherein the pH of the buffer is approximately 3.5.
16. A HPLC method according to any one of the preceding paragraphs, wherein the first liquid A comprises one or more additional solvents.
17. A HPLC method according to paragraph 16, wherein the additional solvent is a substantially water-miscible solvent.
18. A HPLC method according to paragraph 16 or 17, wherein the additional solvent is an organic solvent selected from a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof.
19. A HPLC method according to paragraph 18, wherein the additional solvent is methanol.
20. A HPLC method according to any one of paragraphs 16 to 19, wherein the first liquid A comprises 10 to 90% v/v of the additional solvent.
21. A HPLC method according to paragraph 20, wherein the first liquid A comprises approximately 60% v/v of the additional solvent.
22. A HPLC method according to any one of paragraphs 16 to 21, wherein the additional solvent is the same as the second liquid B.
23. A HPLC method according to any one of the preceding paragraphs, wherein the second liquid B is an organic solvent.
24. A HPLC method according to any one of the preceding paragraphs, wherein the second liquid B is a substantially water-miscible solvent.
25. A HPLC method according to any one of the preceding paragraphs, wherein the second liquid B is a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof.
26. A HPLC method according to any one of the preceding paragraphs, wherein the second liquid B is selected from methanol, ethanol, acetonitrile, n-propanol or iso-propanol, or a mixture thereof
27. A HPLC method according to paragraph 26, wherein the second liquid B is methanol.
28. A HPLC method according to paragraph 27, wherein the first liquid A is a mixture of aqueous potassium dihydrogen phosphate-methanol (40:60 v/v) and the second liquid B is methanol.
29. A HPLC method according to any one of the preceding paragraphs, wherein a mobile phase flow rate of between 0.01 and 10 ml/min is used.
30. A HPLC method according to paragraph 29, wherein a mobile phase flow rate of about 1 ml/min is used.
31. A HPLC method according to any one of the preceding paragraphs, which comprises a gradient programming so that the relative concentration of the liquids A and B is varied to a gradient between 100% A:0% B to 0% A:100% B run over 10 to 180 minutes.
32. A HPLC method according to paragraph 31, wherein the gradient is run over 30 to 120 minutes.
33. A HPLC method according to paragraph 32, wherein the gradient is run over 30 to 60 minutes.
34. A HPLC method according to any one of the preceding paragraphs, wherein the first liquid A is a mixture of 0.02 M aqueous potassium dihydrogen phosphate-methanol (40:60 v/v) and the second liquid B is methanol.
35. A HPLC method according to paragraph 34, wherein the gradient is as follows:

| Time (min) | % A | % B |
|---|---|---|
| 0 | 100 | 0 |
| 5 | 100 | 0 |
| 35 | 50 | 50 |
| 40 | 100 | 0 |
| 50 | 100 | 0 |

36. A HPLC method according to any one of the preceding paragraphs, wherein the stationary phase used is a gel.
37. A HPLC method according to any one of the preceding paragraphs, wherein the stationary phase used is chiral.
38. A HPLC method according to any one of the preceding paragraphs, wherein the mobile phase further comprises a chiral selector.
39. A HPLC method according to any one of the preceding paragraphs, wherein the stationary phase used is reverse phase.
40. A HPLC method according to paragraph 39, wherein the stationary phase used is octadecylsilyl silica gel, octylsilyl silica gel, phenylalkyl silica gel, cyanopropyl silica gel, aminopropyl silica gel or an alkyl-diol silica gel.
41. A HPLC method according to paragraph 40, wherein the stationary phase used is octadecylsilyl silica gel or octylsilyl silica gel.
42. A HPLC method according to paragraph 41, wherein the stationary phase comprises a Sunfire C18 (250 mm×4.6 mm), 5μ column.
43. A HPLC method according to any one of the preceding paragraphs, wherein the stationary phase has a particle size of between 0.1 and 100 μm.
44. A HPLC method according to paragraph 43, wherein the stationary phase has a particle size of about 5 μm.
45. A HPLC method according to any one of the preceding paragraphs, wherein the stationary phase has a pore size of between 10 and 1000 Å.
46. A HPLC method according to any one of the preceding paragraphs, wherein the chromatography is carried out at a temperature between approximately 15 to 40° C.
47. A HPLC method according to any one of the preceding paragraphs, wherein the chromatography is carried out in a column between 10 mm and 5000 mm in length.
48. A HPLC method according to any one of the preceding paragraphs, wherein the chromatography is carried out in a column between 0.01 mm and 100 mm in internal diameter.
49. A HPLC method according to any one of the preceding paragraphs, wherein the eluent is analysed by a detector such as a UV or visible spectrophotometer, a fluorescence spectrophotometer, a differential refractometer, an electrochemical detector, a mass spectrometer, a light scattering detector or a radioactivity detector.
50. A HPLC method according to any one of the preceding paragraphs, wherein the clopidogrel analysed is for use in a pharmaceutical composition.
51. A HPLC method according to any one of the preceding paragraphs, wherein the method is a method of analysing a pharmaceutical composition comprising clopidogrel.
52. A HPLC method according to any one of the preceding paragraphs, wherein the clopidogrel is in the form of a salt, solvate or hydrate.
53. A HPLC method according to paragraph 52, wherein the clopidogrel is either the bisulfate or hydrogen bromide salt.
54. A HPLC method according to any one of the preceding paragraphs, which detects and optionally quantifies one or more impurities selected from:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile.
   55. A HPLC method according to any one of the preceding paragraphs, which detects and optionally quantifies in a single run one or more impurities selected from:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile.
   56. A HPLC method according to any one of the preceding paragraphs, which detects and quantifies in a single run all impurities including those selected from the following compounds:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile.
   57. A HPLC method for analysing clopidogrel, wherein the mobile phase comprises a polar protic organic solvent, and the stationary phase comprises a gel.
   58. A HPLC method according to paragraph 57, wherein the polar protic organic solvent is a substantially water-miscible solvent.
   59. A HPLC method according to paragraph 57 or 58, wherein the polar protic organic solvent is selected from acetic acid, methanol, ethanol, n-propanol, n-butanol, iso-propanol, iso-butanol, sec-butanol or tert-butanol, or a mixture thereof.
   60. A HPLC method according to paragraph 59, wherein the polar protic organic solvent is selected from methanol, ethanol, n-propanol or iso-propanol, or a mixture thereof.
   61. A HPLC method according to paragraph 60, wherein the polar protic organic solvent is methanol.
   62. A HPLC method according to any one of paragraphs 57 to 61, wherein the mobile phase comprises two or more liquids, including a first liquid A and a second liquid B, and wherein the second liquid B comprises or is the polar protic organic solvent.
   63. A HPLC method according to paragraph 62, wherein the first liquid A is aqueous based.
   64. A HPLC method according to paragraph 63, wherein the first liquid A comprises water or an aqueous solution of a buffer.
   65. A HPLC method according to paragraph 64, wherein the buffer is an acid or an organic salt or an inorganic salt.
   66. A HPLC method according to paragraph 65, wherein the buffer is a phosphate salt, an acetate salt, a formate salt or trifluoroacetic acid.
   67. A HPLC method according to paragraph 65 or 66, wherein the buffer is a phosphate salt.
   68. A HPLC method according to paragraph 67, wherein the buffer is potassium dihydrogen phosphate.
   69. A HPLC method according to any one of paragraphs 64 to 68, wherein the buffer is present at a concentration of 0.001 to 0.1 M.
   70. A HPLC method according to paragraph 69, wherein the buffer is present at a concentration of 0.001 to 0.05 M.
   71. A HPLC method according to paragraph 70, wherein the buffer is present at a concentration of 0.005 to 0.05 M.
   72. A HPLC method according to paragraph 71, wherein the buffer is present at a concentration of approximately 0.02 M.
   73. A HPLC method according to paragraph 71, wherein the buffer is potassium dihydrogen phosphate present at a concentration of 0.005 to 0.05 M.
   74. A HPLC method according to paragraph 73, wherein the potassium dihydrogen phosphate is present at a concentration of approximately 0.02 M.
   75. A HPLC method according to any one of paragraphs 64 to 74, wherein the pH of the buffer is approximately 2 to 6.
   76. A HPLC method according to paragraph 75, wherein the pH of the buffer is approximately 3.5.
   77. A HPLC method according to any one of paragraphs 62 to 76, wherein the first liquid A comprises one or more additional solvents.
   78. A HPLC method according to paragraph 77, wherein the additional solvent is a substantially water-miscible solvent.
   79. A HPLC method according to paragraph 77 or 78, wherein the additional solvent is an organic solvent selected from a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof.
   80. A HPLC method according to paragraph 79, wherein the additional solvent is methanol.
   81. A HPLC method according to any one of paragraphs 77 to 80, wherein the first liquid A comprises 10 to 90% v/v of the additional solvent.
   82. A HPLC method according to paragraph 81, wherein the first liquid A comprises approximately 60% v/v of the additional solvent.
   83. A HPLC method according to any one of paragraphs 77 to 82, wherein the additional solvent is the same as the second liquid B.
   84. A HPLC method according to any one of paragraphs 62 to 83, wherein the first liquid A is a mixture of aqueous potassium dihydrogen phosphate-methanol (40:60 v/v) and the second liquid B is methanol.
   85. A HPLC method according to any one of paragraphs 62 to 84, wherein a mobile phase flow rate of between 0.01 and 10 ml/min is used.
   86. A HPLC method according to paragraph 85, wherein a mobile phase flow rate of about 1 ml/min is used.
   87. A HPLC method according to any one of paragraphs 62 to 86, wherein the HLPC method is an isocratic method.
   88. A HLPC method according to paragraph 87, wherein the relative concentration of the liquids A and B is set between 99.5% A:0.5% B and 0.5% A:99.5% B.
   89. A HPLC method according to paragraph 88, wherein the relative concentration of the liquids A and B is about 50% A:50% B.
   90. A HPLC method according to any one of paragraphs 62 to 86, wherein the relative concentration of the liquids of the mobile phase is varied to a predetermined gradient.
   91. A HPLC method according to paragraph 90, which comprises a gradient programming so that the relative concentration of the liquids A and B is varied to a gradient between 100% A:0% B to 0% A:100% B run over 10 to 180 minutes.
   92. A HPLC method according to paragraph 91, wherein the gradient is run over 30 to 120 minutes.
   93. A HPLC method according to paragraph 92, wherein the gradient is run over 30 to 60 minutes.
   94. A HPLC method according to any one of paragraphs 90 to 93, wherein the first liquid A is a mixture of 0.02 M aqueous potassium dihydrogen phosphate-methanol (40:60 v/v) and the second liquid B is methanol.
   95. A HPLC method according to paragraph 94, wherein the gradient is as follows:

| Time (min) | % A | % B |
|---|---|---|
| 0 | 100 | 0 |
| 5 | 100 | 0 |
| 35 | 50 | 50 |
| 40 | 100 | 0 |
| 50 | 100 | 0 |

96. A HPLC method according to any one of paragraphs 57 to 95, wherein the stationary phase used is a silica gel.
97. A HPLC method according to any one of paragraphs 57 to 96, wherein the stationary phase used is chiral.
98. A HPLC method according to any one of paragraphs 57 to 97, wherein the mobile phase further comprises a chiral selector.
99. A HPLC method according to any one of paragraphs 57 to 98, wherein the stationary phase used is reverse phase.
100. A HPLC method according to paragraph 99, wherein the stationary phase used is octadecylsilyl silica gel, octylsilyl silica gel, phenylalkyl silica gel, cyanopropyl silica gel, aminopropyl silica gel or an alkyl-diol silica gel.
101. A HPLC method according to paragraph 100, wherein the stationary phase used is octadecylsilyl silica gel or octylsilyl silica gel.
102. A HPLC method according to paragraph 101, wherein the stationary phase comprises a Sunfire C18 (250 mm×4.6 mm), 5μ column.
103. A HPLC method according to any one of paragraphs 57 to 102, wherein the stationary phase has a particle size of between 0.1 and 100 μm.
104. A HPLC method according to paragraph 103, wherein the stationary phase has a particle size of about 5 μm.
105. A HPLC method according to any one of paragraphs 57 to 104, wherein the stationary phase has a pore size of between 10 and 1000 Å.
106. A HPLC method according to any one of paragraphs 57 to 105, wherein the chromatography is carried out at a temperature between approximately 15 to 40° C.
107. A HPLC method according to any one of paragraphs 57 to 106, wherein the chromatography is carried out in a column between 10 mm and 5000 mm in length.
108. A HPLC method according to any one of paragraphs 57 to 107, wherein the chromatography is carried out in a column between 0.01 mm and 100 mm in internal diameter.
109. A HPLC method according to any one of paragraphs 57 to 108, wherein the eluent is analysed by a detector such as a UV or visible spectrophotometer, a fluorescence spectrophotometer, a differential refractometer, an electrochemical detector, a mass spectrometer, a light scattering detector or a radioactivity detector.
110. A HPLC method according to any one of paragraphs 57 to 109, wherein the clopidogrel analysed is for use in a pharmaceutical composition.
111. A HPLC method according to any one of paragraphs 57 to 110, wherein the method is a method of analysing a pharmaceutical composition comprising clopidogrel.
112. A HPLC method according to any one of paragraphs 57 to 111, wherein the clopidogrel is in the form of a salt, solvate or hydrate.
113. A HPLC method according to paragraph 112, wherein the clopidogrel is either the bisulfate or hydrogen bromide salt.
114. A HPLC method according to any one of paragraphs 57 to 113, which detects and optionally quantifies one or more impurities selected from:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile.
   115. A HPLC method according to any one of paragraphs 57 to 114, which detects and optionally quantifies in a single run one or more impurities selected from:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile.
   116. A HPLC method according to any one of paragraphs 57 to 115, which detects and quantifies in a single run all impurities including those selected from the following compounds:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile.
   117. A method for analysing a substance, comprising the detection and optional quantification of one or more impurities selected from:
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile.
   118. A method according to paragraph 117, further comprising the detection and optional quantification of one or more impurities selected from:
(+)-(S)-(o-chlorophenyl)-6,7-dihydrothieno[3,2-c]pyridine-5(4H)-acetic acid; and
methyl (±)-(o-chlorophenyl)-4,5-dihydrothieno[2,3-c]pyridine-6(7H)-acetate.
   119. A method according to paragraph 117 or 118, wherein the substance is an active pharmaceutical ingredient.
   120. A method according to any one of paragraphs 117 to 119, wherein the substance is clopidogrel.
   121. A method according to paragraph 120, wherein the clopidogrel is in the form of a salt, solvate or hydrate.
   122. A method according to paragraph 121, wherein the clopidogrel is either the bisulfate or hydrogen bromide salt.
   123. A method according to any one of paragraphs 120 to 122, wherein the clopidogrel analysed is for use in a pharmaceutical composition.
   124. A method according to paragraph 117 or 118, wherein the method is a method of analysing a pharmaceutical composition comprising clopidogrel.
   125. A method according to any one of paragraphs 117 to 124, wherein the substance comprises less than 25 wt. % of the one or more impurities.
   126. A method according to any one of paragraphs 117 to 125, wherein the method comprises the use of HPLC.
   127. A method according to paragraph 126, wherein the mobile phase comprises two or more liquids, including a first liquid A and a second liquid B.
   128. A method according to paragraph 127, wherein the first liquid A is aqueous based.
   129. A method according to paragraph 128, wherein the first liquid A comprises water or an aqueous solution of a buffer.
   130. A method according to paragraph 129, wherein the buffer is an acid or an organic salt or an inorganic salt.
   131. A method according to paragraph 130, wherein the buffer is a phosphate salt, an acetate salt, a formate salt or trifluoroacetic acid.
   132. A method according to paragraph 130 or 131, wherein the buffer is a phosphate salt.
   133. A method according to paragraph 132, wherein the buffer is potassium dihydrogen phosphate.
   134. A method according to any one of paragraphs 129 to 133, wherein the buffer is present at a concentration of 0.001 to 0.1 M.
   135. A method according to paragraph 134, wherein the buffer is present at a concentration of 0.001 to 0.05 M.
   136. A method according to paragraph 135, wherein the buffer is present at a concentration of 0.005 to 0.05 M.
   137. A method according to paragraph 136, wherein the buffer is present at a concentration of approximately 0.02 M.
   138. A method according to paragraph 136, wherein the buffer is potassium dihydrogen phosphate present at a concentration of 0.005 to 0.05 M.
   139. A method according to paragraph 138, wherein the potassium dihydrogen phosphate is present at a concentration of approximately 0.02 M.
   140. A method according to any one of paragraphs 129 to 139, wherein the pH of the buffer is approximately 2 to 6.
   141. A method according to paragraph 140, wherein the pH of the buffer is approximately 3.5.
   142. A method according to any one of paragraphs 127 to 141, wherein the first liquid A comprises one or more additional solvents.
   143. A method according to paragraph 142, wherein the additional solvent is a substantially water-miscible solvent.
   144. A method according to paragraph 142 or 143, wherein the additional solvent is an organic solvent selected from a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof
   145. A method according to paragraph 144, wherein the additional solvent is methanol.
   146. A method according to any one of paragraphs 142 to 145, wherein the first liquid A comprises 10 to 90% v/v of the additional solvent.
   147. A method according to paragraph 146, wherein the first liquid A comprises approximately 60% v/v of the additional solvent.
   148. A method according to any one of paragraphs 142 to 147, wherein the additional solvent is the same as the second liquid B.
   149. A method according to any one of paragraphs 127 to 148, wherein the second liquid B is an organic solvent.
   150. A method according to any one of paragraphs 127 to 149, wherein the second liquid B is a substantially water-miscible solvent.
   151. A method according to any one of paragraphs 127 to 150, wherein the second liquid B is a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof.
   152. A method according to paragraph 151, wherein the second liquid B is selected from methanol, ethanol, acetonitrile, n-propanol or iso-propanol, or a mixture thereof
   153. A method according to paragraph 152, wherein the second liquid B is methanol.
   154. A method according to paragraph 153, wherein the first liquid A is a mixture of aqueous potassium dihydrogen phosphate-methanol (40:60 v/v) and the second liquid B is methanol.
   155. A method according to any one of paragraphs 127 to 154, wherein a mobile phase flow rate of between 0.01 and 10 ml/min is used.
   156. A method according to paragraph 155, wherein a mobile phase flow rate of about 1 ml/min is used.
   157. A method according to any one of paragraphs 127 to 156, wherein the HLPC method is an isocratic method.
   158. A method according to paragraph 157, wherein the relative concentration of the liquids A and B is set between 99.5% A:0.5% B and 0.5% A:99.5% B.
   159. A method according to paragraph 158, wherein the relative concentration of the liquids A and B is about 50% A:50% B.
   160. A method according to any one of paragraphs 127 to 156, wherein the relative concentration of the liquids of the mobile phase is varied to a predetermined gradient.
   161. A method according to paragraph 160, which comprises a gradient programming so that the relative concentration of the liquids A and B is varied to a gradient between 100% A:0% B to 0% A:100% B run over 10 to 180 minutes.
   162. A method according to paragraph 161, wherein the gradient is run over 30 to 120 minutes.
   163. A method according to paragraph 162, wherein the gradient is run over 30 to 60 minutes.
   164. A method according to any one of paragraphs 160 to 163, wherein the first liquid A is a mixture of 0.02 M aqueous potassium dihydrogen phosphate-methanol (40:60 v/v) and the second liquid B is methanol.
   165. A method according to paragraph 164, wherein the gradient is as follows:

| Time (min) | % A | % B |
|---|---|---|
| 0 | 100 | 0 |
| 5 | 100 | 0 |
| 35 | 50 | 50 |
| 40 | 100 | 0 |
| 50 | 100 | 0 |

166. A method according to any one of paragraphs 126 to 165, wherein the stationary phase used is a gel.
167. A method according to any one of paragraphs 126 to 166, wherein the stationary phase used is chiral.
168. A method according to any one of paragraphs 126 to 167, wherein the mobile phase further comprises a chiral selector.
169. A method according to any one of paragraphs 126 to 168, wherein the stationary phase used is reverse phase.
170. A method according to paragraph 169, wherein the stationary phase used is octadecylsilyl silica gel, octylsilyl silica gel, phenylalkyl silica gel, cyanopropyl silica gel, aminopropyl silica gel or an alkyl-diol silica gel.
171. A method according to paragraph 170, wherein the stationary phase used is octadecylsilyl silica gel or octylsilyl silica gel.
172. A method according to paragraph 171, wherein the stationary phase comprises a Sunfire C18 (250 mm×4.6 mm), 5μ column.
173. A method according to any one of paragraphs 126 to 172, wherein the stationary phase has a particle size of between 0.1 and 100 μm.
174. A method according to paragraph 173, wherein the stationary phase has a particle size of about 5 μm.
175. A HPLC method according to any one of paragraphs 126 to 174, wherein the stationary phase has a pore size of between 10 and 1000 Å.
176. A method according to any one of paragraphs 126 to 175, wherein the chromatography is carried out at a temperature between approximately 15 to 40° C.
177. A method according to any one of paragraphs 126 to 176, wherein the chromatography is carried out in a column between 10 mm and 5000 mm in length.
178. A method according to any one of paragraphs 126 to 177, wherein the chromatography is carried out in a column between 0.01 mm and 100 mm in internal diameter.
179. A method according to any one of paragraphs 126 to 178, wherein the eluent is analysed by a detector such as a UV or visible spectrophotometer, a fluorescence spectrophotometer, a differential refractometer, an electrochemical detector, a mass spectrometer, a light scattering detector or a radioactivity detector.

The present invention is illustrated but in no way limited by the following example.

### Example

### Experimental Conditions

Column: Sunfire C18 (250 mm×4.6 mm), 5μ, 100 Å pore size;

Flow rate: 1 ml/min;

Detection: 225 nm;

Sample concentration: 1000 ppm;

Diluent: methanol;

First Liquid A: 0.02 M aqueous potassium dibasic hydrogen phosphate (anhydrous)-methanol (40:60 v/v);

Second liquid B: methanol;

Mobile phase: First liquid A-Second liquid B gradient.

The gradient program is described below:

| Time (min) | % A | % B |
|---|---|---|
| 0 | 100 | 0 |
| 5 | 100 | 0 |
| 35 | 50 | 50 |
| 40 | 100 | 0 |
| 50 | 100 | 0 |

Retention times (RT), Relative retention times (RRT), Limit of Detection (LOD) and Limit of Quantitation (LOQ) obtained for all the intermediates and clopidogrel are summarised in Table 1.

TABLE 1
Retention times (RT), Relative retention times (RRT), Limit of Detection (LOD) and Limit of Quantitation (LOQ)

| | Approximate | Approximate | | |
|---|---|---|---|---|
| Component | RT (min) | RRT | LOD (%) | LOQ (%) |
| Clopidogrel (I) | 23.5 | 1.00 | 0.01 | 0.03 |
| (+)-(S)-(o-chlorophenyl)-6,7-dihydrothieno[3,2-c]pyridine-5(4H)- | 4.1 | 0.17 | 0.0075 | 0.0225 |
| acetic acid (listed as Impurity A in USP 29) (II) | | | | |
| methyl (±)-(o-chlorophenyl)-4,5-dihydrothieno[2,3-c]pyridine-6(7H)- | 27.3 | 1.15 | 0.01 | 0.03 |
| acetate (listed as Impurity B in USP 29) (III) | | | | |
| D-(+)-α-4,5,6,7-tetrahydrothieno[3,2-c]-5-pyridyl-(o-chlorophenyl)acetamide | 4.6 | 0.20 | 0.0075 | 0.0225 |
| (IV) | | | | |
| α-4,5,6,7-tetrahydrothieno[3,2-c]-5-pyridyl-(o-chlorophenyl)acetonitrile | 35.6 | 1.50 | 0.0075 | 0.0225 |
| (V) | | | | |

1-179. (canceled)

180. A HPLC method for analysing clopidogrel, wherein the mobile phase comprises two or more liquids, including a first liquid A and a second liquid B, and the relative concentration of the liquids is varied to a predetermined gradient.

181. A HPLC method according to claim 180, wherein the first liquid A is aqueous based.

182. A HPLC method according to claim 181, wherein the first liquid A comprises water or an aqueous solution of a buffer.

183. A HPLC method according to claim 182, wherein:
(i) the buffer is an acid or an organic salt or an inorganic salt; and/or
(ii) the buffer is a phosphate salt, an acetate salt, a formate salt or trifluoroacetic acid; and/or
(iii) the buffer is a phosphate salt; and/or
(iv) the buffer is potassium dihydrogen phosphate; and/or
(v) the buffer is present at a concentration of 0.001 to 0.1 M; and/or
(vi) the buffer is present at a concentration of 0.001 to 0.05 M; and/or
(vii) the buffer is present at a concentration of 0.005 to 0.05 M; and/or
(viii) the buffer is present at a concentration of approximately 0.02 M; and/or
(ix) the buffer is potassium dihydrogen phosphate present at a concentration of 0.005 to 0.05 M; and/or
(x) the buffer is potassium dihydrogen phosphate present at a concentration of approximately 0.02 M; and/or
(xi) the pH of the buffer is approximately 2 to 6; and/or
(xii) the pH of the buffer is approximately 3.5.

184. A HPLC method according to claim 180, wherein the first liquid A comprises one or more additional solvents.

185. A HPLC method according to claim 184, wherein:
(i) the additional solvent is a substantially water-miscible solvent; and/or
(ii) the additional solvent is an organic solvent selected from a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof; and/or
(iii) the additional solvent is methanol; and/or
(iv) the first liquid A comprises 10 to 90% v/v of the additional solvent; and/or
(v) the first liquid A comprises approximately 60% v/v of the additional solvent; and/or
(vi) the additional solvent is the same as the second liquid B.

186. A HPLC method according to claim 180, wherein the second liquid B is:
(i) an organic solvent; and/or
(ii) a substantially water-miscible solvent; and/or
(iii) a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof; and/or
(iv) selected from methanol, ethanol, acetonitrile, n-propanol or iso-propanol, or a mixture thereof; and/or
(v) methanol.

187. A HPLC method according to claim 180, wherein:
(i) the first liquid A is a mixture of aqueous potassium dihydrogen phosphate-methanol (40:60 v/v) and the second liquid B is methanol; and/or
(ii) the first liquid A is a mixture of 0.02 M aqueous potassium dihydrogen phosphate-methanol (40:60 v/v) and the second liquid B is methanol; and/or
(iii) the first liquid A is a mixture of 0.02 M aqueous potassium dihydrogen phosphate-methanol (40:60 v/v) and the second liquid B is methanol, and wherein the gradient is as follows:

| Time (min) | % A | % B |
|---|---|---|
| 0 | 100 | 0 |
| 5 | 100 | 0 |
| 35 | 50 | 50 |
| 40 | 100 | 0 |
| 50 | 100 | 0 |

188. A HPLC method according to claim 180 wherein:
(i) the stationary phase used is a gel; and/or
(ii) the stationary phase used is chiral; and/or
(iii) the mobile phase further comprises a chiral selector; and/or
(iv) the stationary phase used is reverse phase; and/or
(v) the stationary phase used is octadecylsilyl silica gel, octylsilyl silica gel, phenylalkyl silica gel, cyanopropyl silica gel, aminopropyl silica gel or an alkyl-diol silica gel; and/or
(vi) the stationary phase used is octadecylsilyl silica gel or octylsilyl silica gel; and/or
(vii) the stationary phase comprises a Sunfire C18 (250 mm×4.6 mm), 5μ column; and/or
(viii) the stationary phase has a particle size of between 0.1 and 100 μm; and/or
(ix) the stationary phase has a particle size of about 5 μm; and/or
(x) the stationary phase has a pore size of between 10 and 1000 Å; and/or
(xi) a mobile phase flow rate of between 0.01 and 10 ml/min or a mobile phase flow rate of about 1 ml/min is used; and/or
(xii) the method comprises a gradient programming so that the relative concentration of the liquids A and B is varied to a gradient between 100% A:0% B to 0% A:100% B run over 10 to 180 minutes or run over 30 to 120 minutes or run over 30 to 60 minutes; and/or
(xiii) the chromatography is carried out at a temperature between approximately 15 to 40° C.; and/or
(xiv) the chromatography is carried out in a column between 10 mm and 5000 mm in length; and/or
(xv) the chromatography is carried out in a column between 0.01 mm and 100 mm in internal diameter; and/or
(xvi) the eluent is analysed by a detector such as a UV or visible spectrophotometer, a fluorescence spectrophotometer, a differential refractometer, an electrochemical detector, a mass spectrometer, a light scattering detector or a radioactivity detector.

189. A HPLC method according to claim 180, wherein:
(i) the clopidogrel analysed is for use in a pharmaceutical composition; and/or
(ii) the method is a method of analysing a pharmaceutical composition comprising clopidogrel; and/or
(iii) the clopidogrel is in the form of a salt, solvate or hydrate; and/or
(iv) the clopidogrel is either the bisulfate or hydrogen bromide salt.

190. A HPLC method according to claim 180, which:
(i) detects and optionally quantifies one or more impurities selected from:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile; and/or
(ii) detects and optionally quantifies in a single run one or more impurities selected from:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile; and/or
(iii) detects and quantifies in a single run all impurities including those selected from the following compounds:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile.

191. A HPLC method for analysing clopidogrel, wherein the mobile phase comprises a polar protic organic solvent, and the stationary phase comprises a gel.

192. A HPLC method according to claim 191, wherein the polar protic organic solvent is:
(i) a substantially water-miscible solvent; and/or
(ii) selected from acetic acid, methanol, ethanol, n-propanol, n-butanol, iso-propanol, iso-butanol, sec-butanol or tert-butanol, or a mixture thereof; and/or
(iii) selected from methanol, ethanol, n-propanol or iso-propanol, or a mixture thereof; and/or
(iv) methanol.

193. A HPLC method according to claim 191, wherein the mobile phase comprises two or more liquids, including a first liquid A and a second liquid B, and wherein the second liquid B comprises or is the polar protic organic solvent.

194. A HPLC method according to claim 193, wherein the first liquid A is aqueous based.

195. A HPLC method according to claim 194, wherein the first liquid A comprises water or an aqueous solution of a buffer.

196. A HPLC method according to claim 195, wherein:
(i) the buffer is an acid or an organic salt or an inorganic salt; and/or
(ii) the buffer is a phosphate salt, an acetate salt, a formate salt or trifluoroacetic acid; and/or
(iii) the buffer is a phosphate salt; and/or
(iv) the buffer is potassium dihydrogen phosphate; and/or
(v) the buffer is present at a concentration of 0.001 to 0.1 M; and/or
(vi) the buffer is present at a concentration of 0.001 to 0.05 M; and/or
(vii) the buffer is present at a concentration of 0.005 to 0.05 M; and/or
(viii) the buffer is present at a concentration of approximately 0.02 M; and/or
(ix) the buffer is potassium dihydrogen phosphate present at a concentration of 0.005 to 0.05 M; and/or
(x) the buffer is potassium dihydrogen phosphate present at a concentration of approximately 0.02 M; and/or
(xi) the pH of the buffer is approximately 2 to 6; and/or
(xii) the pH of the buffer is approximately 3.5.

197. A HPLC method according to claim 193, wherein the first liquid A comprises one or more additional solvents.

198. A HPLC method according to claim 197, wherein:
(i) the additional solvent is a substantially water-miscible solvent; and/or
(ii) the additional solvent is an organic solvent selected from a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof; and/or
(iii) the additional solvent is methanol; and/or
(iv) the first liquid A comprises 10 to 90% v/v of the additional solvent; and/or
(v) the first liquid A comprises approximately 60% v/v of the additional solvent; and/or
(vi) the additional solvent is the same as the second liquid B.

199. A HPLC method according to claim 193, wherein the HLPC method is:
(i) an isocratic method; and/or
(ii) an isocratic method, and wherein the relative concentration of the liquids A and B is set between 99.5% A:0.5% B and 0.5% A:99.5% B; and/or
(iii) an isocratic method, and wherein the relative concentration of the liquids A and B is about 50% A:50% B.

200. A HPLC method according to claim 193, wherein the relative concentration of the liquids of the mobile phase is:
(i) varied to a predetermined gradient; and/or
(ii) varied to a predetermined gradient, and which comprises a gradient programming so that the relative concentration of the liquids A and B is varied to a gradient between 100% A:0% B to 0% A:100% B run over 10 to 180 minutes or run over 30 to 120 minutes or run over 30 to 60 minutes.

201. A HPLC method according to claim 193, wherein:
(i) the first liquid A is a mixture of aqueous potassium dihydrogen phosphate-methanol (40:60 v/v) and the second liquid B is methanol; and/or
(ii) the first liquid A is a mixture of 0.02 M aqueous potassium dihydrogen phosphate-methanol (40:60 v/v) and the second liquid B is methanol; and/or
(iii) the first liquid A is a mixture of 0.02 M aqueous potassium dihydrogen phosphate-methanol (40:60 v/v) and the second liquid B is methanol, and wherein the gradient is as follows:

| Time (min) | % A | % B |
|---|---|---|
| 0 | 100 | 0 |
| 5 | 100 | 0 |
| 35 | 50 | 50 |
| 40 | 100 | 0 |
| 50 | 100 | 0 |

202. A HPLC method according to claim 191, wherein:
the stationary phase used is a silica gel; and/or
(ii) the stationary phase used is chiral; and/or
(iii) the mobile phase further comprises a chiral selector; and/or
(iv) the stationary phase used is reverse phase; and/or
(v) the stationary phase used is octadecylsilyl silica gel, octylsilyl silica gel, phenylalkyl silica gel, cyanopropyl silica gel, aminopropyl silica gel or an alkyl-diol silica gel; and/or
(vi) the stationary phase used is octadecylsilyl silica gel or octylsilyl silica gel; and/or
(vii) the stationary phase comprises a Sunfire C18 (250 mm×4.6 mm), 5μ column; and/or
(viii) the stationary phase has a particle size of between 0.1 and 100 μm; and/or
(ix) the stationary phase has a particle size of about 5 μm; and/or
(x) the stationary phase has a pore size of between 10 and 1000 Å; and/or
(xi) a mobile phase flow rate of between 0.01 and 10 ml/min or a mobile phase flow rate of about 1 ml/min is used; and/or
(xii) the chromatography is carried out at a temperature between approximately 15 to 40° C.; and/or
(xiii) the chromatography is carried out in a column between 10 mm and 5000 mm in length; and/or
(xiv) the chromatography is carried out in a column between 0.01 mm and 100 mm in internal diameter; and/or
(xv) the eluent is analysed by a detector such as a UV or visible spectrophotometer, a fluorescence spectrophotometer, a differential refractometer, an electrochemical detector, a mass spectrometer, a light scattering detector or a radioactivity detector.

203. A HPLC method according to claim 191, wherein:
(i) the clopidogrel analysed is for use in a pharmaceutical composition; and/or
(ii) the method is a method of analysing a pharmaceutical composition comprising clopidogrel; and/or
(iii) the clopidogrel is in the form of a salt, solvate or hydrate; and/or
(iv) the clopidogrel is either the bisulfate or hydrogen bromide salt.

204. A HPLC method according to claim 191, which:
(i) detects and optionally quantifies one or more impurities selected from:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile; and/or
(ii) detects and optionally quantifies in a single run one or more impurities selected from:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile; and/or
(iii) detects and quantifies in a single run all impurities including those selected from the following compounds:
(+)-(S)-(o-chlorophenyl)-6,7-dihydrothieno[3,2-c]pyridine-5(4H)-acetic acid;
methyl (±)-(o-chlorophenyl)-4,5-dihydrothieno[2,3-c]pyridine-6(7H)-acetate;
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile.

205. A method for analysing a substance, comprising the detection and optional quantification of one or more impurities selected from:
D-(+)-α-4,5,6,7-tetrahydrothieno[3,2-c]-5-pyridyl-(o-chlorophenyl)acetamide; and
α-4,5,6,7-tetrahydrothieno[3,2-c]-5-pyridyl-(o-chlorophenyl)acetonitrile.

206. A method according to claim 205, further comprising the detection and optional quantification of one or more impurities selected from:
(+)-(S)-(o-chlorophenyl)-6,7-dihydrothieno[3,2-c]pyridine-5(4H)-acetic acid; and
methyl (±)-(o-chlorophenyl)-4,5-dihydrothieno[2,3-c]pyridine-6(7H)-acetate.

207. A method according to claim 205, wherein:
(i) the substance is an active pharmaceutical ingredient; and/or
(ii) the substance is clopidogrel; and/or
(iii) the substance is clopidogrel, and wherein the clopidogrel is in the form of a salt, solvate or hydrate; and/or
(iv) the substance is clopidogrel, and wherein the clopidogrel is either the bisulfate or hydrogen bromide salt; and/or
(v) the substance is clopidogrel, and wherein the clopidogrel analysed is for use in a pharmaceutical composition; and/or
(vi) the method is a method of analysing a pharmaceutical composition comprising clopidogrel; and/or
(vii) the substance comprises less than 25 wt. % of the one or more impurities.

208. A method according to claim 205, wherein the method comprises the use of HPLC.

209. A method according to claim 208, wherein the mobile phase comprises two or more liquids, including a first liquid A and a second liquid B.

210. A method according to claim 209, wherein the first liquid A is aqueous based.

211. A method according to claim 210, wherein the first liquid A comprises water or an aqueous solution of a buffer.

212. A method according to claim 211, wherein:
(i) the buffer is an acid or an organic salt or an inorganic salt; and/or
(ii) the buffer is a phosphate salt, an acetate salt, a formate salt or trifluoroacetic acid; and/or
(iii) the buffer is a phosphate salt; and/or
(iv) the buffer is potassium dihydrogen phosphate; and/or
(v) the buffer is present at a concentration of 0.001 to 0.1 M; and/or
(vi) the buffer is present at a concentration of 0.001 to 0.05 M; and/or
(vii) the buffer is present at a concentration of 0.005 to 0.05 M; and/or
(viii) the buffer is present at a concentration of approximately 0.02 M; and/or
(ix) the buffer is potassium dihydrogen phosphate present at a concentration of 0.005 to 0.05 M; and/or
(x) the buffer is potassium dihydrogen phosphate present at a concentration of approximately 0.02 M; and/or
(xi) the pH of the buffer is approximately 2 to 6; and/or
(xii) the pH of the buffer is approximately 3.5.

213. A method according to claim 209, wherein the first liquid A comprises one or more additional solvents.

214. A method according to claim 213, wherein:
(i) the additional solvent is a substantially water-miscible solvent; and/or
(ii) the additional solvent is an organic solvent selected from a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof; and/or
(iii) the additional solvent is methanol; and/or
(iv) the first liquid A comprises 10 to 90% v/v of the additional solvent; and/or
(v) the first liquid A comprises approximately 60% v/v of the additional solvent; and/or
(vi) the additional solvent is the same as the second liquid B.

215. A method according to claim 209, wherein the second liquid B is:
(i) an organic solvent; and/or
(ii) a substantially water-miscible solvent; and/or
(iii) a polar protic solvent such as acetic acid, methanol, ethanol, n-propanol, n-butanol, iso-propanol, iso-butanol, sec-butanol or tert-butanol, or a dipolar aprotic solvent such as tetrahydrofuran, acetone, dimethoxyethane, DMF, DMSO, 1,4-dioxane, pyridine or acetonitrile, or a mixture thereof; and/or
(iv) selected from methanol, ethanol, acetonitrile, n-propanol or iso-propanol, or a mixture thereof; and/or
(v) methanol.

216. A method according to claim 209, wherein the HLPC method is:
(i) an isocratic method; and/or
(ii) an isocratic method, and wherein the relative concentration of the liquids A and B is set between 99.5% A:0.5% B and 0.5% A:99.5% B; and/or
(iii) an isocratic method, and wherein the relative concentration of the liquids A and B is about 50% A:50% B.

217. A method according to claim 209, wherein the relative concentration of the liquids of the mobile phase is:
(i) varied to a predetermined gradient; and/or
(ii) varied to a predetermined gradient, and which comprises a gradient programming so that the relative concentration of the liquids A and B is varied to a gradient between 100% A:0% B to 0% A:100% B run over 10 to 180 minutes or run over 30 to 120 minutes or run over 30 to 60 minutes.

218. A method according to claim 209, wherein:
(i) the first liquid A is a mixture of aqueous potassium dihydrogen phosphate-methanol (40:60 v/v) and the second liquid B is methanol; and/or
(ii) the first liquid A is a mixture of 0.02 M aqueous potassium dihydrogen phosphate-methanol (40:60 v/v) and the second liquid B is methanol; and/or
(iii) the first liquid A is a mixture of 0.02 M aqueous potassium dihydrogen phosphate-methanol (40:60 v/v) and the second liquid B is methanol, and wherein the gradient is as follows:

| Time (min) | % A | % B |
|---|---|---|
| 0 | 100 | 0 |
| 5 | 100 | 0 |
| 35 | 50 | 50 |
| 40 | 100 | 0 |
| 50 | 100 | 0 |

219. A method according to claim 208, wherein:
(i) the stationary phase used is a gel; and/or
(ii) the stationary phase used is chiral; and/or
(iii) the mobile phase further comprises a chiral selector; and/or
(iv) the stationary phase used is reverse phase; and/or
(v) the stationary phase used is octadecylsilyl silica gel, octylsilyl silica gel, phenylalkyl silica gel, cyanopropyl silica gel, aminopropyl silica gel or an alkyl-diol silica gel; and/or
(vi) the stationary phase used is octadecylsilyl silica gel or octylsilyl silica gel; and/or
(vii) the stationary phase comprises a Sunfire C18 (250 mm×4.6 mm), 5μ column; and/or
(viii) the stationary phase has a particle size of between 0.1 and 100 μm; and/or
(ix) the stationary phase has a particle size of about 5 μm; and/or
(x) the stationary phase has a pore size of between 10 and 1000 Å; and/or
(xi) a mobile phase flow rate of between 0.01 and 10 ml/min or a mobile phase flow rate of about 1 ml/min is used; and/or
(xii) the chromatography is carried out at a temperature between approximately 15 to 40° C.; and/or
(xiii) the chromatography is carried out in a column between 10 mm and 5000 mm in length; and/or
(xiv) the chromatography is carried out in a column between 0.01 mm and 100 mm in internal diameter; and/or
(xv) the eluent is analysed by a detector such as a UV or visible spectrophotometer, a fluorescence spectrophotometer, a differential refractometer, an electrochemical detector, a mass spectrometer, a light scattering detector or a radioactivity detector.
